(19) United States
(12) Patent Application Publication
(10) Pub. No.: US 2025/0073363 A1
(43) Pub. Date: Mar. 6, 2025

(54) Aircraft Sanitization System

(71) Applicant: HEATHER MARIE STRAW, Everett, WA (US)

(72) Inventor: HEATHER MARIE STRAW, Everett, WA (US)

(21) Appl. No.: 18/457,260

(22) Filed: Aug. 28, 2023

(51) Int. Cl. A61L 2/22; A61L 2/26

(52) U.S. Cl. CPC A61L 2/22; A61L 2/26; A61L 2202/15; A61L 2202/25

(57) ABSTRACT

Using the existing cabin air flow/heating/air conditioning system lines or running lines in the same path the system would disperse a nontoxic sanitizer through overhead personal air flow nozzle. In existing aircraft, the air nozzles would need to be converted to run both the air and sanitizer, the sanitizer will need a wide flow misting spray.

A main sanitizer canister would install in a small convenient location to connect to existing air system lines. Upon passenger and crew departure of the cabin the sanitizer would be dispersed, then air would be run to dry the lines and assist with drying the seats. Sanitizer canister can be small and refilled with each flight or a larger canister if weight isn't a concern.

### BACKGROUND

This is a system that can be retrofitted and/or installed in new commercial or private aircraft of any make and model. This will enable, to the best of the airline's ability, to keep every inch of the inside of the cabin sanitized minimizing the spread of viruses. Greatly reducing the spread of viruses during air travel will increase confidence in air travel now as well as if there is another pandemic type of outbreak. Therefore, this system will protect revenue during a viral pandemic. This system would also reduce human labor time during the cleaning process saving commercial airline business in labor expenses.

### SUMMARY

An Aircraft Sanitization System to protect human health as well as airline carriers bottom line.

### FIGURES

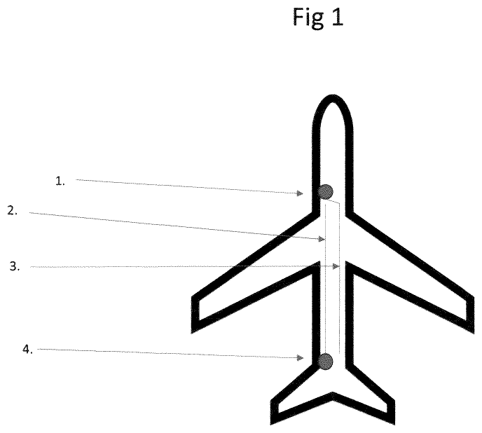

FIG. 1
1. This is an option of where the canister of sanitizer could be located.
2. Lines to run the sanitizer through left side
3. Lines to run the sanitizer through left side
4. Another option for canister location

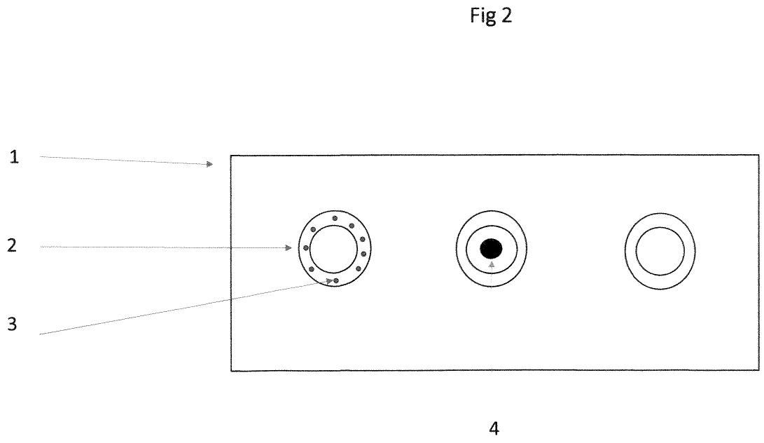

FIG. 2
1. Aircraft existing air system
2. Aircraft airflow device
3. Sanitizer spray option 1
4. Sanitizer spray option 2

### SPECIFICATION

By either using the existing cabin air flow/heating/air conditioning system (FIG. 2, 1) lines (FIGS. 1, 2 & 3) or running lines (FIGS. 1, 2 & 3) in the same path the system would disperse a fast drying, nontoxic sanitizer through overhead personal air flow nozzle (FIG. 2, 2&3). In existing aircraft, the air nozzles would likely need to be swapped out with a version that can run both the air and sanitizer, the sanitizer would need to have a wide flow misting spray. The sanitizer may be able to run in the existing airflow ducts. If not, new lines (FIG. 1, 2&3) could run through the existing ducts using the existing air ducts as conduit.

There will be a main sanitizer canister (FIGS. 1, 1 & 4) located at the existing site of the heating and air conditioning units. Once the plane lands and has been cleared, the sanitizer would be dispersed for a predetermined amount of time, long enough for full coverage and sanitization. Then air would be run for a predetermined amount of time to dry the lines and assist with the drying process on the seats, etc. Once the sanitizer is run it would be refilled for the next flight if keeping the aircraft weight down and space is a concern. Otherwise, a larger canister can be installed to cover the sanitization of multiple flights.

The attached drawings are just samples of how I envision this product to be installed given I have no aircraft engineering experience. The examples are only general representations of the implementation of the system.

1. Enable airline's ability to keep every inch of the inside of the cabin sanitized.

2. Minimize the spread of viruses and bacteria.

3. Minimizing the spread of viruses and bacteria will increase confidence in travelers.

4. Increasing confidence in travelers will protect revenue during another pandemic or even flu season.

5. This system will reduce humane labor saving on labor expenses.

6. Reducing human labor will also reduce human error.
